(19) United States
(12) Patent Application Publication
(10) Pub. No.: US 2011/0229625 A1
(43) Pub. Date: Sep. 22, 2011

(54) Transgenic Forage Crops with Enhanced Nutrition

(76) Inventors: William Ralph Hiatt, Davis, CA (US); Yun-Jeong Hong, Davis, CA (US)

(21) Appl. No.: 13/029,500

(22) Filed: Feb. 17, 2011

Related U.S. Application Data
(60) Provisional application No. 61/305,350, filed on Feb. 17, 2010.

(51) Int. Cl. A23K 1/00; A01H 1/02; C12N 15/82; A01H 5/00

(52) U.S. Cl. 426/635; 800/260; 800/278; 800/284; 800/263; 800/298

(57) ABSTRACT

The present invention provides a method to modify a forage crop to exhibit enhanced animal feed nutrition. The forage crop is genetically modified to provide increased levels of phenolic compounds and polyphenol oxidases. The invention provides methods, compositions, plants, plant cells, seeds, plant parts, processes forage and commodity products with enhanced animal feed nutrition.

### CROSS-REFERENCE TO RELATED APPLICATIONS

This application claims priority from U.S. Provisional Application Ser. No. 61/305,350 (filed Feb. 17, 2010), the entire text of which is incorporated herein by reference.

### FIELD OF THE INVENTION

The present invention relates to transgenic forage crops with enhanced nutrition as animal feed. The invention generally relates to plant genetic engineering and the improvement of the nutritional components of the plant. The invention relates to a transgenic forage crop with a recombinant DNA construct that reduces expression or the activity of an enzyme in the lignin biosynthetic pathway and a DNA construct that provides for the expression of a polyphenol oxidase. The transgenic forage crop exhibits increased levels of phenolic acids and increased levels of o-quinones as manifested by dark pigment formation (browning) that enhances the levels of antioxidant molecules and reduces proteolysis. The invention also relates to plants, plant parts, plant seeds, plant cells, agricultural products, and methods related to enhancing the nutrition of a forage crop.

### BACKGROUND OF THE INVENTION

Forage crops that include legumes, grasses and brassicas are grown throughout the world to provide an animal feed that is high in protein. There is a need in animal feed to provide antioxidant molecules and enzymes that produce o-quinones that complex with proteins in the feed and reduce proteolysis during silage of the feed. The need to reduce proteolysis is especially important for dairy cattle feed. Alfalfa (Medicago sativa) is a forage legume and often comprises twenty-three to thirty-four percent of dairy cattle feed. Alfalfa is low in both phenolic acids that can serve as a substrate for a polyphenol oxidase and the polyphenol oxidase enzyme that would provide o-quinones that enhance protein stability in the feed. Hence, alfalfa protein is poorly utilized by ruminant animals resulting in loss of protein during ensilage and degradation in the cow rumen and high levels of excretion of excess nitrogen in the urine. The excretion of the excess nitrogen into the environment from dairy cattle is a significant source of water and air pollution. There is a need to improve protein utilization and feed value of alfalfa to enhance the nutrition of animal feed containing alfalfa or other forage crops and reduce the levels of nitrogen waste compounds in the environmental.

Current methods to reduce proteolysis in forage feed rely upon, for example, the incorporation of various proteolytic enzyme inhibitors, modifying the pH, adding phenolic compounds, or adding polyphenol oxidase enzymes into the feed. The present invention provides a method and compositions through genetic engineering wherein a transgenic forage plant has increased endogenous increased levels of phenolic compounds and polyphenol oxidase enzyme activity that produces the o-quinones and dark pigments during storage, ensilage, processing and feeding that reduces proteolysis of the feed protein allowing it to more available to the feed animal for nutrition.

### SUMMARY OF THE INVENTION

A method to enhance the nutrition of a forage plant comprising the steps of transforming a forage plant cell with a first DNA construct that provides for the suppression of expression or activity of an enzyme of the lignin biosynthetic pathway; regenerating the plant cell into a whole plant; selecting the whole plant that exhibits increased levels of phenolic compounds; transforming a cell of the plant with a second DNA construct that provides for expression of a polyphenol oxidase and regenerating the plant cell into a whole plant or breeding said plant with a second plant comprising said second DNA construct; selecting a plant or progeny of said breeding, wherein extracts of said plant or progeny produces an elevated level of a dark colored pigment relative to a forage plant not comprising the first and second DNA constructs.

In one aspect of the invention is a forage plant comprising a DNA construct that suppresses the activity of an enzyme in the lignin biosynthetic pathway and a DNA construct expressing a polyphenol oxidase.

In another aspect of the invention the enzyme of the lignin biosynthetic pathway is selected from the group consisting of trans-caffeoyl-CoA 3-O-methyltransferase, caffeic acid 3-O-methyltransferase, hydroxycinnamoyl CoA: quinate/shikimat hydroxycinnamoyl transferase, coumarate 3-hydroxylase, and ferulate 5-hydroxylase.

In another aspect of the invention is an alfalfa plant comprising a first DNA construct that suppresses the activity of an enzyme in the lignin biosynthetic pathway and a second DNA construct expressing a polyphenol oxidase.

In another aspect of the invention is an animal feed comprising a forage plant, plant part or seed comprising a DNA construct that suppresses the activity of an enzyme in the lignin biosynthetic pathway and a DNA construct expressing a polyphenol oxidase.

In another aspect of the invention is an animal feed comprising an alfalfa plant, plant part or seed comprising a first DNA construct that suppresses the activity of an enzyme in the lignin biosynthetic pathway and a second DNA construct expressing a polyphenol oxidase, wherein the feed exhibits reduced proteolysis relative to the feed not comprising the DNA constructs.

### DETAILED DESCRIPTION

The invention provides a method to increase the production of o-quinones and tissue browning in forage crops in need of the increase. Forage crops, for example, including but not limited to Sainfoin, Lespedeza, Kura clover, Birdsfoot trefoil, Cicer milkvetch, Crown Vetch and alfalfa.

The following definitions and methods are provided to better define the present invention and to guide those of ordinary skill in the art in the practice of the present invention. Unless otherwise noted, terms are to be understood according to conventional usage by those of ordinary skill in the relevant art.

As used herein, the term “forage crop” means a forage legume, forage grass or forage brassica and includes all plant varieties that can be bred with the forage crop, including related wild forage species.

Alfalfa (Medicago sativa) is a forage legume often used for animal feed, especially dairy cattle.

As used herein, the term “comprising” means “including but not limited to”.

The present invention provides DNA molecules and their corresponding nucleotide sequences. As used herein, the term “DNA”, “DNA molecule”, “polynucleotide molecule” refers to a double-stranded DNA molecule of genomic or synthetic origin, i.e., a polymer of deoxyribonucleotide bases or a polynucleotide molecule, read from the 5′ (upstream) end to the 3′ (downstream) end. As used herein, the term “DNA sequence”, “nucleotide sequence” or “polynucleotide sequence” refers to the nucleotide sequence of a DNA molecule. The nomenclature used herein is that required by Title 37 of the United States Code of Federal Regulations §1.822 and set forth in the tables in WIPO Standard ST.25 (1998), Appendix 2, Tables 1 and 3.

“DNA Construct” or “recombinant DNA molecule” refers to a combination of heterologous DNA genetic elements in operable linkage that is often used to provide new traits to a recipient organism. As used herein, the term “recombinant” refers to a form of DNA and/or protein and/or an organism that would not normally be found in nature and as such was created by human intervention. Such human intervention may produce a recombinant DNA molecule and/or a recombinant plant. As used herein, a “recombinant DNA molecule” is a DNA molecule comprising a combination of DNA molecules that would not naturally occur together and is the result of human intervention, e.g., a DNA molecule that is comprised of a combination of at least two DNA molecules heterologous to each other, and/or a DNA molecule that is artificially synthesized and comprises a polynucleotide sequence that deviates from the polynucleotide sequence that would normally exist in nature. As used herein, a “recombinant plant” is a plant that would not normally exist in nature, is the result of human intervention, and contains a transgene and/or recombinant DNA molecule incorporated into its genome.

“Operably Linked”. A first nucleic-acid sequence is “operably” linked with a second nucleic-acid sequence when the first nucleic-acid sequence is placed in a functional relationship with the second nucleic-acid sequence. For example, a promoter is operably linked to a protein-coding sequence if the promoter effects the transcription or expression of the coding sequence. Generally, operably linked DNA sequences are contiguous and, where necessary to join two protein-coding regions, in reading frame.

The term “promoter” or “promoter region” refers to a polynucleic acid molecule that functions as a regulatory element, usually found upstream (5′) to a coding sequence, that controls expression of the coding sequence by controlling production of messenger RNA (mRNA) by providing the recognition site for RNA polymerase and/or other factors necessary for start of transcription at the correct site. As contemplated herein, a promoter or promoter region includes variations of promoters derived by means of ligation to various regulatory sequences, random or controlled mutagenesis, and addition or duplication of enhancer sequences. The promoter region disclosed herein, and biologically functional equivalents thereof, are responsible for driving the transcription of coding sequences under their control when introduced into a host as part of a suitable recombinant vector, as demonstrated by its ability to produce mRNA.

As used herein, the term “transgene” refers to a polynucleotide molecule artificially incorporated into a host cell's genome. Such transgene may be heterologous to the host cell. The term “transgenic plant” refers to a plant comprising such a transgene.

“Regeneration” refers to the process of growing a plant from a plant cell (e.g., plant protoplast or explant).

“Transformation” refers to a process of introducing an exogenous polynucleic acid molecule (e.g., a DNA construct, a recombinant polynucleic acid molecule) into a cell or protoplast and that exogenous polynucleic acid molecule is incorporated into a host cell genome or an organelle genome (e.g., chloroplast or mitochondria) or is capable of autonomous replication.

“Transformed” or “transgenic” refers to a cell, tissue, organ, or organism into which a foreign polynucleic acid, such as a DNA vector or recombinant polynucleic acid molecule. A “transgenic” or “transformed” cell or organism also includes progeny of the cell or organism and progeny produced from a breeding program employing such a “transgenic” plant as a parent in a cross and exhibiting an altered phenotype resulting from the presence of the foreign polynucleic acid molecule.

The term “transgene” refers to any polynucleic acid molecule normative to a cell or organism transformed into the cell or organism. “Transgene” also encompasses the component parts of a native plant gene modified by insertion of a normative polynucleic acid molecule by directed recombination or site specific mutation.

“Transit peptide” or “targeting peptide” molecules. These terms generally refer to peptide molecules that when linked to a protein of interest directs the protein to a particular tissue, cell, subcellular location, or cell organelle. Examples include, but are not limited to, chloroplast transit peptides, nuclear targeting signals, and vacuolar signals. The chloroplast transit peptide is of particular utility in the present invention to direct expression of the PPO enzyme to the chloroplast.

Plants of the present invention may pass along the recombinant DNA to a progeny. As used herein, “progeny” includes any plant, seed, plant cell, and/or regenerable plant part comprising the recombinant DNA derived from an ancestor plant. Plants, progeny, and seeds may be homozygous or heterozygous for a transgene. In practicing the present invention, two different transgenic plants can be crossed to produce hybrid offspring that contain two independently segregating heterologous genes. Selfing of appropriate progeny can produce plants that are homozygous for both genes. Back-crossing to a parental plant and out-crossing with a non-transgenic plant are also contemplated, as is vegetative propagation. Descriptions of other methods that are commonly used for different traits and crops can be found in one of several references, e.g., Fehr, in Breeding Methods for Cultivar Development, Wilcox J. ed., American Society of Agronomy, Madison Wis. (1987).

The plants and seeds used in the methods disclosed herein may also contain one or more additional transgenes. Such transgene may be any nucleotide sequence encoding a protein or RNA molecule conferring a desirable trait including but not limited to increased insect resistance, increased water use efficiency, increased yield performance, increased drought resistance, increased seed quality, improved nutritional quality, and/or increased herbicide tolerance, in which the desirable trait is measured with respect to a forage plant lacking such additional transgene.

Polyphenol oxidase (PPO) is a type-3 copper protein which catalyzes the oxidation of monophenols or to o-diphenols and then to o-quinones. PPO-generated quinones are highly reactive, and will crosslink with proteins or polymerize, generating dark-colored tannins and melanins. In intact plant cells, plastid localized PPO is physically separated from its phenolic substrates. Thus, PPO activity is generally observed only upon loss of cellular compartmentalization caused by senescence, wounding, or other tissue damage. Phenolic acids are simple compounds such as caffeic acid, vanillin, and courmaric acid. Phenolic compounds also form a diverse group that includes the widely distributed hydroxybenzoic and hydroxycinnamic acids (p-coumaric, caffeic acid, ferulic acid), and tannins. Tannins in forage plants have been shown to reduce protein degradation, increase microbial protein synthesis, and increase the efficiency of protein utilization. Phenolic compounds seem to be universally distributed in plants. They have been the subject of a great number of chemical, biological, agricultural, and medical studies. Many polyphenol oxidases are known, for example, including but not limited to those described in U.S. Pat. No. 7,449,617, (the sequences disclosed therein are herein incorporated by reference and consisting of SEQ ID NO: 10 is the amino acid sequence from walnut, SEQ ID NO: 48 is the amino acid sequence from N. crassa, SEQ ID NO: 52 is the amino acid sequence from R. thomasiana, SEQ ID NO: 53 is the amino acid sequence from V. faba, SEQ ID NO: 54 is the amino acid sequence from M. domestica, SEQ ID NO: 55 is the amino acid sequence from I. batatas, SEQ ID NO: 56 is the amino acid sequence from L. esculentum, SEQ ID NO: 57 is the amino acid sequence from S. tuberosum, SEQ ID NO: 58 is the amino acid sequence from L. esculentum, SEQ ID NO: 59 is the amino acid sequence from S. oleracea and SEQ ID NO: 60 is the amino acid sequence from J. regia).

Chloroplast transit peptides (CTPs) are engineered to be fused to the N terminus of a prokaryote PPO to direct the enzyme into the plant chloroplast. In the native plant PPOs, chloroplast transit peptide regions are contained in the native coding sequence. The native CTP may be substituted with a heterologous CTP during construction of a transgene plant expression cassette. Many chloroplast-localized proteins are expressed from nuclear genes as precursors and are targeted to the chloroplast by a chloroplast transit peptide (CTP) that is removed during the import steps. Examples of other such chloroplast proteins include the small subunit (SSU) of Ribulose-1,5-bisphosphate carboxylase (rubisco), Ferredoxin, Ferredoxin oxidoreductase, the light-harvesting complex protein I and protein II, and Thioredoxin F. It has been demonstrated in vivo and in vitro that non-chloroplast proteins may be targeted to the chloroplast by use of protein fusions with a CTP and that a CTP sequence is sufficient to target a protein to the chloroplast. For example, incorporation of a suitable chloroplast transit peptide, such as, the Arabidopsis thaliana EPSPS CTP (Klee et al., Mol. Gen. Genet. 210:437-442 (1987), and the Petunia hybrida EPSPS CTP (della-Cioppa et al., Proc. Natl. Acad. Sci. USA 83:6873-6877 (1986) has been shown to target heterologous EPSPS protein sequences to chloroplasts in transgenic plants. Those skilled in the art will recognize that various chimeric constructs can be made that utilize the functionality of a particular CTP to import glyphosate resistant EPSPS enzymes into the plant cell chloroplast.

### Lignin Biosynthetic Pathway Enzymes

The lignin pathway starts with the conversion of phenylalanine to cinnamate by phenylalanine ammonia lyase (PAL). The second reaction is performed by cinnamate 4-hydroxylase (C4H) which converts cinnamate to 4-coumarate. These two enzymes form the core of the phenylpropanoid pathway including lignin biosynthesis. Other enzymes in the pathway include C3H or 4-coumarate 3-hydroxylase, which converts 4-coumaroyl shikimate or quinate to caffeoyl shikimate or quinate; HCT, hydroxycinnamoyl CoA: hydroxycinnamoyl transferase which acts at two places catalyzing the formation of 4-coumaroyl shikimate (or quinate), the substrate for C3H, from 4-Coumaroyl CoA, and also acting in the opposite direction on caffeoyl shikimate (or quinate), to yield caffeoyl CoA. CCoAOMT (trans-caffeoyl-CoA 3-O-methyltransferase) converts caffeoyl CoA to feruloyl CoA and might also be involved in other reactions. COMT (caffeic acid O-methyl transferase) acts on 5-hydroxy coniferaldehyde and converts it into sinapaldehyde. Ferulate 5-hydroxylase (F5H) converts coniferaldehyde to 5-hydroxyconiferaldehyde. Key enzymes of the pathway in which down regulation could result in the accumulation of phenolic compounds include but are not limited to trans-caffeoyl-CoA 3-O-methyltransferase (CCoAOMT or CCOMT), caffeic acid 3-O-methyltransferase, hydroxycinnamoyl CoA: quinate/shikimat hydroxycinnamoyl transferase, coumarate 3-hydroxylase, and ferulate 5-hydroxylase. The sequences of the lignin biosynthetic pathway enzymes are disclosed in US20070079398 and incorporated herein by reference.

### Expression of DNA Constructs in Plants

DNA constructs are made that contain various genetic elements necessary for the expression of noncoding and coding sequences in plants. Promoters, leaders, introns, transit peptide encoding polynucleic acids, 3′ transcriptional termination regions are all genetic elements that may be operably linked by those skilled in the art of plant molecular biology to provide a desirable level of expression or functionality.

A variety of promoters specifically active in vegetative tissues, such as leaves, stems, roots and tubers, can be used to express the PPO enzyme and down regulate the lignin biosynthetic pathway enzymes. Plant virus promoter, for example, the CaMV 35S promoter (U.S. Pat. No. 5,352,605, herein incorporated by reference) and the Figwort mosaic virus promoter (U.S. Pat. No. 6,051,753, herein incorporated by reference) express well in many plant species and tissues. Examples of leaf-specific promoters include, but are not limited to the ribulose biphosphate carboxylase (RBCS or RuBISCO) promoters (see, e.g., Matsuoka et al., Plant J. 6:311-319, 1994); the light harvesting chlorophyll a/b binding protein gene promoter (see, e.g., Shiina et al., Plant Physiol. 115:477-483, 1997; Casal et al., Plant Physiol. 116:1533-1538, 1998); and the Arabidopsis thaliana myb-related gene promoter (Atmyb5) (Li et al., FEBS Lett. 379:117-121, 1996).

The “3′ non-translated sequences” means DNA sequences located downstream of a structural nucleotide sequence and include sequences encoding polyadenylation and other regulatory signals capable of affecting mRNA processing or gene expression. The polyadenylation signal functions in plants to cause the addition of polyadenylate nucleotides to the 3′ end of the mRNA precursor. The polyadenylation sequence can be derived from the natural gene, from a variety of plant genes, or from T-DNA. An example of the polyadenylation sequence is the nopaline synthase 3′ sequence (nos 3′; Fraley et al., Proc. Natl. Acad. Sci. USA 80: 4803-4807, 1983). The use of different 3′ non-translated sequences is exemplified by Ingelbrecht et al., Plant Cell 1:671-680, 1989.

The laboratory procedures in recombinant DNA technology used herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al., Molecular Cloning—A Laboratory Manual, 2nd. ed., Cold Spring Harbor Laboratory, Cold Spring Harbor, N.Y. (1989), herein referred to as Sambrook et al., (1989).

Polynucleic acid molecules of interest may also be synthesized, either completely or in part, especially where it is desirable to provide modifications in the polynucleotide sequences, by well-known techniques as described in the technical literature, see, e.g., Carruthers et al., Cold Spring Harbor Symp. Quant. Biol. 47:411-418 (1982), and Adams et al., J. Am. Chem. Soc. 105:661 (1983), both of which are herein incorporated by reference in their entireties. Thus, all or a portion of the polynucleic acid molecules of the present invention may be synthesized using a codon usage table of a selected plant host.

The DNA construct of the present invention may be introduced into the genome of a desired plant host by a variety of conventional transformation techniques that are well known to those skilled in the art. Methods of transformation of plant cells or tissues include, but are not limited to Agrobacterium mediated transformation method and the Biolistics or particle-gun mediated transformation method. Suitable plant transformation vectors for the purpose of Agrobacterium mediated transformation include those derived from a Ti plasmid of Agrobacterium tumefaciens, as well as those disclosed, e.g., by Herrera-Estrella et al., Nature 303:209 (1983); Bevan, Nucleic Acids Res. 12: 8711-8721 (1984); Klee et al., Bio-Technology 3(7): 637-642 (1985). In addition to plant transformation vectors derived from the Ti or root-inducing (Ri) plasmids of Agrobacterium, alternative methods can be used to insert the DNA constructs of this invention into plant cells. Such methods may involve, but are not limited to, for example, the use of liposomes, electroporation, chemicals that increase free DNA uptake, free DNA delivery via microprojectile bombardment, and transformation using viruses or pollen.

The following examples are included to demonstrate examples of certain preferred embodiments of the invention. It should be appreciated by those of skill in the art that the techniques disclosed in the examples that follow represent approaches the inventors have found function well in the practice of the invention, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

### Example 1

Phenolic acid analysis of transgenic alfalfa expressing a DNA construct for the suppression of expression of an enzyme in the lignin biosynthetic pathway. Alfalfa cells were transformed with a DNA construct comprising a DNA segment complimentary to a trans-caffeoyl-CoA 3-O-methyltransferase (CCOMT) coding sequence in order to downregulate the expression of the enzyme. The alfalfa cells were regenerated into whole plants and the plant tissues assayed for the presence of phenolic compounds by and LC/MS method.

Alfalfa tissues (lower stem, upper stem and leaf) were collected in the field and frozen immediately under the liquid nitrogen. Frozen tissues were ground using the Mega-Grinder into fine homogenous powders and followed by lyophilization. Ground lyophilized samples were stored at −80° C. freezer until needed for analysis. The sample was weighed around 200 miligram (mg)±5 mg into a 7 milliliter (ml) amber glass vial with a screw cap after the frozen sample came to room temperature. Phenolic acids were extracted using 10 ml of 100 percent methanol for 72 hours (hrs) in a cold room with the presence of BHT (butylated hydroxytoluene) (spiked 20 microliter (0) of 10 microgram/milliliter (μg/ml) stock solution) as an antioxidant and 3-hydroxycoumarin (spiked 200 of 10 μg/ml stock solution) as an internal standard to all the samples before the extraction. After 72 hrs, the samples were centrifuged and supernatants were transferred to new 7 ml amber glass vials. The extracts then were evaporated to dryness under nitrogen and reconstituted in 1 ml of 50 percent methanol in water, followed by addition of 1 ml chloroform to remove chlorophyll. After vortexing vigorously, the phase separation was done by centrifuge. The upper layer was transferred to a new amber vial and evaporated to dryness. 2000 of 20 percent methanol in water was added to a dried extract and filtrated through 0.2 micrometer (μm) PTFE (polytetrafluoroethylene) micro-centrifugal filters. The final filtered sample was transferred to LC/MS vials and injected to HPLC-PDA/ESI-MS. HPLC-PDA/ESI-MS/MS analysis—20 μl per sample was injected to HPLC by the Waters Acquity autosampler and monitored by PDA detector. The eluent was continuously sprayed onto Q trap through ESI probe and scanned by using MRM scan mode.

### HPLC/PDA Conditions

HPLC: Waters Acquity HPLC

Column: cBEH (C₁₈) HPLC column (2.1×100 mm, 1.7 m)

PDA detector: scanned from 190 to 550 nm

Conditions for mobile solvents: Gradient applied using two mobile solvents
Solvent A=10 milimolar (mM) ammonium formate (pH adjusted to 4 by formic acid) in 5 percent acetonitrile in water
Solvent B=10 mM ammonium formate (pH adjusted to 4 by formic acid) in 50 percent acetonitrile in water
Gradient

TABLE 1
UPLC conditions

| | Flow rate | |
|---|---|---|
| Time (min) | (ml/min) | B % |
| 0 | 0.3 | 0 |
| 19 | 0.3 | 19 |
| 20 | 0.3 | 100 |
| 28 | 0.3 | 100 |
| 29 | 0.3 | 0 |
| 35 | 0.3 | 0 |

TABLE 2
Mass Spectrometer Conditions - Mass
spectrometer: ABI 4000 Q trap

| Parameters | Values |
|---|---|
| Curtain Gas | 40 |
| IS (Ion Spray, Voltage) | −4500 |
| Temparature (° C.) | 550 |
| Gas 1 | 50 |
| Gas 2 | 60 |
| Cad Gas | Medium |
| Entrance Potential (Voltage) | −10 |

TABLE 3
Conditions for MRM transitions

| | | Rt | *Q1 | *Q3 | | | |
|---|---|---|---|---|---|---|---|
| | Metabolite ID | (min) | (da) | (da) | *DP | *EP | *CE |
| 1 | caffeoyl glucose | 5.42 | 341.02 | 179 | −75 | −10 | −22 |
| 2 | caffeyl alcohol | 8.14 | 164.93 | 101 | −65 | −10 | −30 |
| 3 | caffeyl aldehyde | 13.41 | 162.9 | 134.9 | −65 | −10 | −24 |
| 4 | caffeic acid | 9.36 | 178.89 | 135 | −55 | −10 | −24 |
| 5 | coumaryl alcohol | 12.07 | 149.1 | 103 | −55 | −10 | −26 |
| 6 | coumaric acid | 13.86 | 162.9 | 119.1 | −50 | −10 | −22 |
| 7 | cinnamic acid | 31 | 146.94 | 102.8 | −45 | −10 | −16 |
| 8 | coniferyl alcohol | 9.36 | 178.89 | 88.9 | −55 | −10 | −58 |
| 9 | coniferyl aldehyde | 21.5 | 176.9 | 133.7 | −40 | −10 | −30 |
| 10 | sinapyl alcohol | 16.18 | 208.95 | 161 | −55 | −10 | −24 |
| 11 | sinapyl aldehyde | 23.1 | 206.94 | 149 | −45 | −10 | −34 |
| 12 | ferulic acid | 16.9 | 192.84 | 133.7 | −50 | −10 | −24 |
| 13 | 5OH coniferyl alcohol | 9.36 | 195.1 | 150.9 | −60 | −10 | −28 |
| 14 | 5OH coniferyl | 15.3 | 192.84 | 150 | −50 | −10 | −30 |
| | aldehyde | | | | | | |
| 15 | sinapic acid | 18.1 | 223 | 149 | −60 | −10 | −30 |
| 16 | coumaryl aldehyde | 17.6 | 146.9 | 103.9 | −60 | −10 | −34 |
| 17 | Vanillyl aldehyde | 12 | 151 | 92 | −55 | −10 | −25 |
| 18 | Salicylic acid | 8.26 | 137 | 93 | −55 | −10 | −25 |
| 19 | 3-Hydroxycoumarin | 12.03 | 161 | 151 | −60 | −10 | −35 |
| | (IS) | | | | | | |

*Q1: Mass focused at the first quadrupole, Q3: Mass focused at the third quadrupole, DP: declustering potential, EP: entrance potential, CE: collision energy.

Four transgenic alfalfa events and a nontransgenic alfalfa plant were extracted for phenolic compounds. The four alfalfa events contain a DNA construct for the suppression of expression of the CCOMT enzyme in the lignin biosynthetic pathway. The values were converted to microMolar/kilogram dry weight (μM/kg DW) for caffeic acid for all the events including control. The other o-diphenols including caffeoyl alcohol, caffeoyl aldehyde, and caffeoyl glucose which can be deglycosylated and converted to caffeic acid by plants were expressed as peak area since the absolute quantitation could not be made on these metabolites. Surprisingly, the results shown in Table 4 demonstrated that all four of the transgenic events had substantial increases in caffeic acid, caffeoyl alcohol, caffeoyl aldehyde and caffeoyl glucose.

TABLE 4
Increased levels of phenolic compounds in CCOMT transgenic alfalfa tissues.

| Tissue types | Units | Phenolics | Control | 1 | 2 | 3 | 4 |
|---|---|---|---|---|---|---|---|
| Lower Stem | (μM/kg DW) | Caffeic acid | 1.76 | 34.00 | 36.21 | 35.65 | 36.79 |
| | Peak area | Caffeoyl alcohol | 31252 | 51497 | 78029 | 67863 | 132663 |
| | | Caffeoyl aldehyde | 17895 | 154327 | 140701 | 115319 | 140513 |
| | | Caffeoyl glucose | 837678 | 140035314 | 130537806 | 95513485 | 64375980 |
| Upper stem | (μM/kg DW) | Caffeic acid | 2.32 | 150.65 | 143.36 | 96.76 | 94.36 |
| | Peak area | Caffeoyl alcohol | 34444 | 326210 | 434107 | 178521 | 569616 |
| | | Caffeoyl aldehyde | 8677 | 102803 | 122640 | 70886 | 189797 |
| | | Caffeoyl glucose | 1075160 | 103209189 | 184658567 | 84760145 | 114887601 |
| Leaf | (μM/kg DW) | Caffeic acid | 2.32 | 12.88 | 12.09 | 7.13 | 8.88 |
| | Peak area | Caffeoyl alcohol | 7224.1 | 21219 | 19850.4 | 17103.1 | 12775.4 |
| | | Caffeoyl aldehyde | 1413.84 | 8206.6 | 5129.79 | 1943.7 | 4750.84 |
| | | Caffeoyl glucose | 344692 | 8119277 | 5758128 | 5660712 | 3939297 |

### Example 2

The same transgenic CCOMT alfalfa plants events 1, 2, 3 and 4 were extracted for phenolic compounds and the extracts tested to determine if the increased levels of caffeic acid or other diphenolic compounds that accumulate in CCOMT down regulated alfalfa tissues could be oxidized by the enzyme polyphenol oxidase (PPO). PPO oxidation of phenolics in planta will lead to slower proteolysis of protein in forage harvested and stored. More protein from alfalfa hay would then be available to the animal making this a premium product for ranchers/dairy users.

Sample preparation: Tissues from upper stems of control and the CCOMT alfalfa events were collected in the field and frozen immediately with liquid nitrogen. Frozen tissues were ground using the Mega-Grinder into fine homogenous powders, lyophilized and stored at −80° C. until needed for analysis. Approximately 300 mg of tissue was extracted with 10 ml of 100 percent methanol for 72 hrs at 4° C. Samples were then centrifuged and the supernatants were evaporated to dryness under nitrogen, reconstituted in 100 μl of 50 percent methanol in water and filtrated through 0.2 μm PTFE micro-centrifugal filters.

Solutions: Polyphenol oxidase (I.U.B.: 1.14.18.1, monophenol,dihydroxyphenlyalanine: O₂ oxidoreductase) from mushroom was purchased from Worthington Biochemicals (Lakewood N.J., USA). Approximately 1 mg of enzyme was dissolved it in 1 ml of water (1460 U/ml), and then further diluted by adding 275 μl of the solution to 725 μl of water to give a working solution of ˜400 U/ml. 0.5 M Phosphate buffer was prepared by adding 6.8 g in 100 ml and adjusting the pH 6.5 with 5N KOH. A 100 mM stock solution of caffeic acid was prepared by dissolving 12 mg in of 650 ul methanol. Buffer solutions for enzyme assays were prepared by adding 10 ml of 0.5 M phosphate buffer with 9.5 ml of water (−PPO) or 10 ml of 0.5 M phosphate buffer with 8.5 ml of water and 1 ml of the PPO (400 U/ml) working solution (+PPO).

Enzyme Assay: 25 ul of the sample extract to be tested were added to either 975 ul of the (−PPO) buffer mix or the (+PPO) buffer mix. Samples were mixed at room temperature. Standards of 0, 18, 90 or 180 μg of caffeic acid were made by mixing 1, 5 or 10 μl of 100 mM caffeic acid with 1 ml of the (+PPO) buffer mix. All samples were incubated for 18 hours at room temperature and absorbance at 475 nm was measured, Absorbance values without PPO added were subtracted from absorbance with PPO to give net caffeic acid equivalent values.

Results: The upper stem samples of events 4 and 2 showed a definite browning after 10 minutes. All samples were stored overnight at room temperature to maximize color formation. The absorbance readings shown in Table 5 are the results after an 18 hr incubation. Surprisingly, all four transgenic events demonstrated an increased absorbance relative to the control indicating that the increased levels of phenolic compounds in the CCOMT plants are substrates for the PPO enzyme.

TABLE 5
Absorbance after 18 hours incubation

| Sample | Absorbance value | Fold increase |
|---|---|---|
| 1 | 0.011 | |
| 1 + PPO | 0.038 | 3.45 |
| 2 | 0.031 | |
| 2 + PPO | 0.115 | 3.71 |
| 3 | 0.036 | |
| 3 + PPO | 0.071 | 1.97 |
| 4 | 0.003 | |
| 4 + PPO | 0.062 | 20.67 |
| Control | 0.033 | |
| Control + PPO | 0.055 | 1.67 |

Having illustrated and described the principles of the present invention, it should be apparent to persons skilled in the art that the invention can be modified in arrangement and detail without departing from such principles. We claim all modifications that are within the spirit and scope of the appended claims. All publications and published patent documents cited in this specification are hereby incorporated by reference to the same extent as if each individual publication or patent application is specifically and individually indicated to be incorporated by reference.

We claim:

1. A method to enhance the nutrition of a forage plant comprising the steps of: 1) transforming a forage plant cell with a first DNA construct that provides for the suppression of expression or activity of an enzyme of the lignin biosynthetic pathway; 2) regenerating said plant cell into a whole plant; 3) selecting said whole plant that exhibits increased levels of a phenolic compound; 4) transforming a cell of said plant with a second DNA construct that provides for expression of a polyphenol oxidase (PPO) and regenerating said plant cell into a whole plant or breeding said plant with a second plant comprising said second DNA construct; 5) selecting a plant or a progeny of said breeding comprising the first and second DNA constructs, wherein an extract of said plant or progeny produces an elevated level of a dark colored pigment relative to a forage plant not comprising the first and second DNA constructs.

2. The method of claim 1, wherein said enzyme of the lignin biosynthetic pathway is selected from the group consisting of trans-caffeoyl-CoA 3-O-methyltransferase, caffeic acid 3-O-methyltransferase, hydroxycinnamoyl CoA: quinate/shikimat hydroxycinnamoyl transferase, coumarate 3-hydroxylase, and ferulate 5-hydroxylase.

3. The method of claim 1, wherein said first DNA construct comprises a promoter molecule that functions in plants cells operably linked to a DNA molecule comprising a effective length of nucleic acid sequence complimentary to a DNA molecule encoding an enzyme of the lignin biosynthetic pathway selected from the group consisting of trans-caffeoyl-CoA 3-O-methyltransferase, caffeic acid 3-O-methyltransferase, hydroxycinnamoyl CoA: quinate/shikimat hydroxycinnamoyl transferase, coumarate 3-hydroxylase, and ferulate 5-hydroxylase.

4. The method of claim 1, wherein said polyphenol oxidase is selected from the group consisting of a walnut PPO, N. crassa PPO, R. thomasiana PPO, V. faba PPO, M. domestica PPO, I. batatas PPO, L. esculentum PPO, S. tuberosum PPO, S. oleracea PPO and J. regia PPO.

5. The method of claim 1, wherein said second DNA construct comprises a promoter molecule that functions in plants cells operably linked to a DNA molecule encoding a polyphenol oxidase enzyme selected from the group consisting of a walnut PPO, N. crassa PPO, R. thomasiana PPO, V. faba PPO, M. domestica PPO, I. batatas PPO, L. esculentum PPO, S. tuberosum PPO, S. oleracea PPO and J. regia PPO.

6. The method of claim 1, wherein said phenolic compound comprises caffeic acid, caffeoyl alcohol, caffeoyl aldehyde or caffeoyl glucose.

7. The method of claim 1, wherein said forage plant is selected from the group consisting of forage legume, forage grass and forage brassica.

8. The method of claim 7, wherein said forage legume is selected from the group consisting of alfalfa, white clover, sainfoin, lespedeza, kura clover, birdsfoot trefoil, cicer milkvetch, and crown vetch.

9. The method of claim 7, wherein said forage grass is selected from the group consisting of tall fescue, meadow fescue and timothy.

10. A forage plant, plant parts or seed produced by the method of claim 1.

11. An animal feed comprising the forage plant, plant parts or seed of claim 10.

12. An animal feed comprising an alfalfa plant, plant part or seed, said alfalfa plant comprising a first DNA construct that suppresses the activity of an enzyme in the lignin biosynthetic pathway and a second DNA construct expressing a polyphenol oxidase, wherein the feed exhibits reduced proteolysis relative to alfalfa in the feed not comprising the DNA constructs.

13. An alfalfa plant comprising a first DNA construct comprising a promoter molecule that functions in plants cells operably linked to a DNA molecule comprising a effective length of nucleic acid sequence complimentary to the DNA molecule encoding an enzyme of the lignin biosynthetic pathway selected from the group consisting of trans-caffeoyl-CoA 3-O-methyltransferase, caffeic acid 3-O-methyltransferase, hydroxycinnamoyl CoA: quinate/shikimate hydroxycinnamoyl transferase, coumarate 3-hydroxylase, and ferulate 5-hydroxylase and a second DNA construct comprising a promoter molecule that functions in plants cells operably linked to a DNA molecule encoding a polyphenol oxidase enzyme, wherein an extract of said plant produces an elevated level of a dark colored pigment relative to an plant not comprising the first and second DNA constructs.
